(19) United States
(12) Patent Application Publication
(10) Pub. No.: US 2016/0346223 A1
(43) Pub. Date: Dec. 1, 2016

(54) INDIVIDUALLY-PACKAGED WIPES FOR ENHANCING SEXUAL INTERCOURSE

(71) Applicant: Aspen Park Pharmaceuticals, New York, NY (US)

(72) Inventor: HARRY FISCH, New York, NY (US)

(73) Assignee: Aspen Park Pharmaceuticals, New York, NY (US)

(21) Appl. No.: 15/114,620

(22) PCT Filed: Jan. 20, 2015
(86) PCT No.: PCT/US15/11961

§ 371 Date: Jul. 27, 2016

Related U.S. Application Data
(60) Provisional application No. 61/932,470, filed on Jan. 28, 2014.

(51) Int. Cl. A61K 9/70; A61K 31/245; A61K 31/167; A61K 9/00

(52) U.S. Cl. CPC A61K 9/7007; A61K 9/0034; A61K 31/245; A61K 31/167

(57) ABSTRACT

The present invention relates to a sealed package containing one wipe, wherein the one wipe comprises a carrier and an active ingredient impregnated therein, wherein the active ingredient is selected from the group consisting of penile desensitizing agents. The sealed package can be opened, the wipe removed and wiped on the penis of a male to reduce the occurrence of premature ejaculation during subsequent intercourse.

### BACKGROUND OF THE INVENTION

1. Field of the Invention

The present invention relates to wipes for enhancing sexual intercourse and to methods of using the wipes to enhance a sexual encounter.

2. Description of Related Art

The American Urological Association defines premature ejaculation as ejaculation that occurs sooner than desired, either before or shortly after penetration, causing distress to either one or both partners (Guideline on the Pharmacologic Management of Premature Ejaculation, American Urological Association, 2004), Premature ejaculation is among the most prevalent of male sexual complaints. A survey has estimated that premature ejaculation affects approximately 25% to 30% of men of all ages (see, Laumann et al., JAMA, 281: 537-544 (1999)). Persistent premature ejaculation can leave sexual partners feeling unsatisfied or unfulfilled, can cause stress or embarrassment to the affected male individual, and can lead to a deterioration of the intimate relationship between partners.

Treatments for premature ejaculation include application of topical anesthetics, e.g., preparations containing benzocaine or a lidocaine-prilocaine combination, to the penis to diminish sensitivity and delay ejaculation. However, these are typically sprayed and, if applied in the area where sexual intercourse is to occur, can leave the area wet and uncomfortable.

WO 2006/022683 describes stimulant/desensitizer swabs for use in preventing premature ejaculation. A pharmaceutical composition including, for example, benzocaine or lidocaine as desensitizer is applied directly to the penis using a cotton swab. The stimulant will stimulate the penis to erection while the desensitizer will delay ejaculation. The applicator is cumbersome to use, not discrete at all, and, moreover, it takes time for the pharmaceutical composition to flow through the applicator to be available for application to the penis.

There remains a need for means of reducing the incidence of premature ejaculation that are less cumbersome, more discrete and allow for instant application of the active ingredient to the penis.

### SUMMARY OF THE INVENTION

These and other objects were met with the present invention, which relates in a first embodiment to a sealed package containing one wipe, said one wipe comprising a carrier and an active ingredient impregnated therein, wherein said active ingredient is selected from the group consisting of penile desensitizing agents.

The present invention relates in a second embodiment to a method of reducing the incidence of premature ejaculation in a male about to engage in sexual intercourse, the method comprising providing a sealed package according to the invention, wherein the sealed package comprises a carrier and a penile desensitizing agent impregnated therein, opening the package, removing the wipe from the package and wiping the wipe on the penis of said male.

### DETAILED DESCRIPTION OF THE INVENTION

As used herein the phrase “containing one wipe” means that the sealed package contains exactly one wipe and no other wipes. Thus, the phrase is not met by a reference showing a sealed package containing multiple wipes unless each of the wipes is individually wrapped, in which case, the individual wrapping constitutes the “sealed package containing one wipe.” A sealed package that contains multiple wipes, wherein each of the wipes is individually wrapped within its own individual sealed package, is within the present invention.

The phrase “penile desensitizing agent” means any chemical substance that when topically applied to the penis of a male who suffers from premature ejaculation delays but does not prevent ejaculation during subsequent sexual intercourse.

In one embodiment, the wipe comprises a penile desensitizing agent. Preferably, the penile desensitizing agent is selected from the group consisting of benzocaine, lidocaine and prilocaine. The concentration of the penile desensitizing agent in the wipes can vary, depending on the particular penile desensitizing agent and standards of practice in the art.

In a most preferred embodiment, the penile desensitizing agent is benzocaine.

In a second most preferred embodiment, the penile desensitizing agent is lidocaine.

In a third most preferred embodiment, the penile desensitizing agent is a combination of lidocaine and prilocaine.

In the most preferred embodiment, the penile desensitizing agent is benzocaine, which is in the wipe in a concentration of 3-7.5% in a water-soluble base.

The wipe may also comprise erection stimulants, for example, as set forth in WO 2006/022683, the relevant contents of which are incorporated herein by reference.

The carrier can be any suitable material. In one embodiment, the carrier is selected from the group consisting of paper and woven and nonwoven fabrics.

In a preferred embodiment, the carrier material is preferably a non-woven material, generally made of synthetic compounds. However, woven materials as well as the use of natural compounds in either woven or nonwoven materials are within the scope of the present invention. The texture and material of the wipe are of high relevance to the performance of the wipe. In one embodiment of the present invention the non-woven material comprises fibers selected from the group consisting of polyolefin, polyester, cellulose, rayon, polyamides, polyesteramide, polyvinyl alcohols, and combinations thereof. The substrate usable for this invention can be manufactured via any suitable process, such as but not limited to, spunlace process and preferably has a dry basis weight of between about 45 grams per square meter (gsm) and 100 gsm, more preferably between 45 gsm and 75 gsm.

In the most preferred embodiment, the carrier is a mixture of rayon and polyolefin, preferably polypropylene, and the penile desensitizing agent is benzocaine.

The packaging itself may be any suitable material, for example, paper, foils, or plastics, as well as any combination thereof, such as foil-lined paper, plastic-lined paper or a wax-lined paper. The package may take a variety of forms such as rectangular, oval, etc. The package may be provided with a separation structure, such as an edge tear area, a zipper-locked edge area, or an openable, adhesively sealed edge area.

### EXAMPLE

A male suffering frequent premature ejaculation is provided with a sealed package containing a wipe comprising a thermal bonded fabric carrier of 75% rayon and 25% polypropylene carrier, for example, Novonette Pattern #2, a thermal bonded fabric available from Ahlstrom Nonwoven LLC. Bethune, S.C., USA. The carrier is impregnated with a solution containing 4% benzocaine, water and ethyl alcohol. Prior to sexual intercourse, the male opens the package, removes the wipe and wipes his penis with the wipe thereby topically transferring the benzocaine solution to his penis. During subsequent sexual intercourse, the benzocaine desensitizes the man's penis, prolonging the normal time for him between first penetration and ejaculation.

While the present invention has been described in conjunction with the specific embodiments set forth above, many alternatives, modifications and other variations thereof will be apparent to those of ordinary skill in the art All such alternatives, modifications and variations are intended to fall within the spirit and scope of the present invention.

1. A sealed package containing one wipe, said one wipe comprising a carrier and an active ingredient impregnated therein, wherein said active ingredient is selected from the group consisting of penile desensitizing agents.

2. The sealed package according to claim 1, wherein the penile desensitizing agent is selected from the group consisting of benzocaine, lidocaine and prilocaine.

3. The sealed package according to claim 2, wherein the penile desensitizing agent is benzocaine.

4. The sealed package according to claim 2, wherein the penile desensitizing agent is lidocaine.

5. The sealed package according to claim 2, wherein the penile desensitizing agent is a combination of lidocaine and prilocaine.

6. The sealed package according to claim 1, wherein the carrier is selected from the group consisting of paper and woven and nonwoven fabrics.

7. The sealed package according to claim 6, wherein the carrier is a non-woven fabric material comprising fibers selected from the group consisting of polyolefin, polyester, cellulose, rayon, polyamides, polyesteramide, polyvinyl alcohols, and combinations thereof.

8. The sealed package according to claim 2, wherein the carrier is a nonwoven fabric comprising rayon and polypropylene fibers and the penile desensitizing agent is benzocaine.

9. A method of reducing the incidence of premature ejaculation in a male about to engage in sexual intercourse, said method comprising providing a sealed package according to claim 1, opening the package, removing the wipe from the package and wiping the wipe on the penis of said male.

10. A sealed package containing one wipe, said one wipe comprising a carrier and either a single active ingredient or a single active ingredient composition, wherein the single active ingredient or single active ingredient composition is the only active ingredient in the wipe, wherein the single active ingredient is selected from the group consisting of penile desensitizing agents, and wherein the single active ingredient composition is selected from the group consisting of mixtures of penile desensitizing agents with erection stimulants.

11. A sealed package containing one wipe, said one wipe comprising a carrier and either a single active ingredient or a single active ingredient composition, wherein the single active ingredient or single active ingredient composition is the only active ingredient in the wipe, wherein the single active ingredient is selected from the group consisting of benzocaine, lidocaine and prilocaine, and wherein the single active ingredient composition is selected from the group consisting of mixtures of at least two of benzocaine, lidocaine and prilocaine.
